(19) United States
(12) Patent Application Publication
(10) Pub. No.: US 2023/0181805 A1
(43) Pub. Date: Jun. 15, 2023

(54) CATHETER FOR INTRAVASCULAR OXYGEN SUPPLEMENTATION

(71) Applicants: Frank H. Boehm, JR., New Hartford, NY (US); Janice Stone, New Hartford, NY (US)

(72) Inventors: Frank H. Boehm, JR., New Hartford, NY (US); Janice Stone, New Hartford, NY (US)

(21) Appl. No.: 17/548,144

(22) Filed: Dec. 10, 2021

(51) Int. Cl. A61M 1/28; A61M 1/32

(52) U.S. Cl. CPC A61M 1/285; A61M 1/32; A61M 2205/3337

(57) ABSTRACT

The invention involves a unique, useful, novel and nonobvious device through which the blood of a patient afflicted with ARDS can have its O₂ level augmented while reducing the CO₂ level. This is achieved by disclosing a unique, useful, novel and nonobvious complex intravascular catheter which creates a system of intravascular, exo-pulmonary oxygenation of the blood.

### BACKGROUND OF THE INVENTION

Adult Respiratory Distress Syndrome (ARDS) is a major cause of mortality and morbidity in the world today. This is a syndrome which is often seen in any major physiologic insult, but is especially treacherous in overwhelming sepsis, massive trauma, major cardiovascular catastrophes such as extensive myocardial infarction or major cerebral infarction, and most recently, has been the principal pathology leading to death in patients infected with Coronavirus, in these settings. It is characterized by diffuse bilateral pulmonary injury with ultimate collapse of the alveolar mechanism and consequential failure to be able to adequately present sufficient amounts of Oxygen (O₂) to the bloodstream for uptake; this hypoxemia initiates a cascade of complications which, in turn, can precipitate multi-organ failure that leads to a terminal event. The patient often succumbs despite respiratory interventions such as ventilator assistance with high O₂ concentrations, and other interventions. For even mild cases, there is a 27% mortality rate, with this increasing to nearly 50% in severe cases. There is clearly a need for better treatment.

The mortality and morbidity of ARDS largely relates to the manner by which the normal pulmonary functions of oxygen and carbon dioxide exchange. A cursory review of cardiopulmonary function and Oxygen transport will be helpful in better appreciating the invention.

At sea level, air is comprised of 78.09% molecular Nitrogen (N₂); 20.95% molecular Oxygen (O₂); 0.93% Argon, 0.04% Carbon Dioxide (CO₂), and trace amounts of other gases.

As tissues require and use O₂ for metabolism and release CO₂ as a byproduct, metabolism, venous blood returning to the heart from all parts of the body has a low level of O₂ and a high level of CO₂. Blood enters into the right side of the heart and is pumped into the lungs, where CO₂ is released in exchange for O₂. The blood with the highest O₂ levels is then returned to the left side of the heart and from there to all parts of the body.

In the healthy individual, inspired air fills the lungs, which are composed of millions of microscopic air sacs known as alveoli. Each alveolus is surrounded by a network of capillaries through which blood cells slowly pass, taking full advantage of membranes lining the alveoli, which are thin and quite permeable to the gases which comprise the atmosphere, particularly molecular Nitrogen (N₂), Oxygen (O₂) and Carbon Dioxide (CO₂). When dissolved in a complex, non-Newtonian liquid such as blood, these concentrations are best represented by the partial pressures (given in torr or mmHg) of each component. N₂ is relatively inert, and does not participate in any metabolic processes, so even though the partial pressure of N₂ in the blood is high, it does not change from arterial blood to venous blood.

In order to drive O₂ exchange, the RBC’s are provided with a unique mechanism. Hemoglobin, which is an iron-rich protein found in all mammalian erythrocytes or red blood cells, has a very high affinity for O₂, and under normal physiologic conditions is central to the mechanism through which O₂ is delivered to the tissues of all such organisms. O₂ is fundamental to the metabolic functions of every cell, and without a sufficient amount of O₂, mammalian cells cannot sustain life and will rapidly die, leading to multiorgan failure and death of the organism itself.

It is important to note that in ARDS, there is no evidence of dysfunction of the hemoglobin itself or the hemoglobin uptake and release mechanism. This assumption is a critical part of the invention.

The most significant complications in ARDS arise from failing to properly oxygenate the body’s tissues. However, the buildup of CO₂ also takes its toll, principally by eventually altering the acid- base balance and the complications associated therewith.

ARDS represents a massive injury to lung tissues, causing the alveoli to become filled with inflammatory fluids, become bruised, collapse and become non-functional. The pathophysiology leading to this cascade of events is beyond the scope of this discussion; the major challenge in ARDS is to be able to maintain an acceptable oxygen level for a long enough time to allow the lungs to recover and heal, usually about 1-2 weeks. Maintaining the patient on high O₂ and ventilation pressures with mechanical ventilation is currently the cornerstone of management; however, this treatment itself is damaging to the lungs, and probably establishes a “vicious cycle,” which in the end makes it more difficult for the lungs to heal and begin to function normally.

In the case of infants with Infant Respiratory Distress Syndrome/IRDS a number of strategies and technologies have been proposed and implemented. The one which has been most commonly used in clinical practice is referred to as “Extra-Corporeal Membrane Oxygenation,” (ECMO). This involves cannulating a blood vessel, diverting blood from the intravascular space through a circuit outside the body where it can be oxygenated and re-introduced to the vascular space through a separately cannulated blood vessel.

The Heart-Lung machine used during cardiac surgery is certainly the most widely used example of Extra-Corporeal oxygenation, combining that with the fact that this machine also mechanically circulates the blood. This is only necessary, or course, in patients whose heart has been temporarily arrested, and would be disadvantageous in patients with a functioning cardiovascular system.

ECMO represented a somewhat less technically complex iteration of this, certainly without the need for a pumping action, and has been very helpful in the treatment of infants with severe respiratory distress. However, for reasons that are somewhat unclear, the results using this method for ARDS have been disappointing, to say the least, complicated in part by issues including sepsis, mechanical injury to the RBC’s, and other problems. Now, this method is only occasionally attempted in that setting.

Various other strategies have been attempted to manage adults through this extreme insult. One strategy which was investigated starting in the 1980′s was the use of IV fluids such as perfluorocarbons (PFC which themselves can carry O₂, and some studies with these were promising. However, these approaches do not circumnavigate the major issue, which is getting the O₂ into the bloodstream to begin with.

In another attempt to address this problem, the art taught by Parker in US 5,706,830 proposed introduction of oxygenated liquid into the bronchus of a patient was not reduced to practice. Although this may have some theoretical foundation, it still does not resolve the challenge of escorting O₂ through the damaged alveoli and into the capillaries, where it can be initially absorbed into the plasma, and ultimately taken up by the hemoglobin in the erythrocytes (also known as the “red blood cells / RBC’s).

There is a need for a device and method for use which can increase the oxygen levels in the blood of patients suffering from ARDS. Ideally, this would be done by introducing O₂ into the intravascular space for direct uptake by the plasma and RBC’s. Such a device would be unique, useful, novel and nonobvious.

### BRIEF DESCRIPTION OF THE INVENTION

The invention involves a unique, useful, novel and nonobvious device through which the blood of a patient afflicted with ARDS can have its O₂ level augmented while reducing the CO₂ level. This is achieved by disclosing a unique, useful, novel and nonobvious complex intravascular catheter which creates a system of intravascular, exo-pulmonary oxygenation of the blood.

This brief description of the invention is intended only to provide a brief overview of subject matter disclosed herein according to one or more illustrative embodiments and does not serve as a guide to interpreting the claims or to define or limit the scope of the invention, which is defined only by the claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale; emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings, in which:

FIG. 1 is a schematic illustration of the cardiopulmonary system, along with examples of where gas exchange normally occurs.

FIG. 2 exhibits the EXPO system including catheter, connection tubing and regulator.

FIG. 3 illustrates a top view of the outer sheath of the catheter.

FIG. 4 portrays a lateral view of an alternative embodiment outer sheath.

FIG. 5 displays a lateral view of the preferred embodiment of the exchange chamber.

FIG. 6 reveals a schematic demonstration of a lateral view of a section of the catheter showing the flow of gases between the blood and the different components of the catheter.

FIG. 7 is a transaxial view showing the schematic demonstration of the gas exchange.

FIG. 8 exhibits a lateral view of an alternative embodiment of the exchange chamber within the catheter.

FIG. 9 displays a transaxial view of the alternative embodiment of the exchange chamber shown in FIG. 8.

FIG. 10 illustrates an elevational view of yet another alternative embodiment of the exchange chamber within the outer sheath.

FIG. 11 shows an elevational perspective of an embodiment of the exchange chamber characterized by multiple elongated cylindrical baffles.

FIG. 12 portrays a transaxial view of the catheter and the exchange chamber in FIG. 11.

FIG. 13 illustrates an elevational view of an alternative embodiment of the catheter in which balloons positioned at the ends of the catheter intermittently interrupt the blood flow along the course of the catheter, thus exposing the momentarily stagnated blood to a prolonged period of gas exchange.

FIG. 14 is a schematic detail of the regulator from a top view.

FIGS. 15A-G portray the preferred method of inserting the catheter into a blood vessel.

FIG. 16 exhibits some of the preferred sites/vessels for placement of the catheter to achieve exopulmonary reoxygenation of the blood.

### SUMMARY OF THE INVENTION

The invention takes full advantage of the hemoglobin mechanism, and that deoxygenated blood, whether within the arterial or venous system, has the high affinity for O₂ and readily exchanges this for CO₂. The lower affinity for CO₂ drives this to even more quickly move from the venous vascular space to air at normal atmospheric partial pressures, or any gaseous admixture with little or nor CO₂. Ultimately, this establishes an intravascular, exo pulmonary oxygenation (EXPO) apparatus, and hereinafter, the invention shall be referred to as the EXPO.

To accomplish this, disclosed herein is a complex intravascular catheter (the EXPO catheter) which comprises two elements: an outer sheath which is a modified intravascular catheter, and an exchange chamber configured to be positioned within the outer sheath/catheter.

The outer sheath, known hereinafter as the sheath catheter is provided with a leading end, a central shaft, and a trailing end. It is, additionally, fabricated from Teflon, polyurethane, or any other substance known and acceptable to the art. In the preferred embodiment, the outer sheath would be a large bore catheter specifically designed to be positioned within a large vessel. Ideally, this would be one of the main venous structures leading to the heart such as the Superior or Inferior Vena Cava (SVC/IVC). The reasoning substantiating this proposal will be discussed below.

Furthermore, in the preferred embodiment, the sheath catheter would be provided with multiple fenestrations, which would permit the blood (RBC’s) passing along the external surface of the catheter to be exposed and in direct contact with the external surface of the exchange chamber, which is positioned within the catheter.

In an alternative embodiment, the sheath catheter may be a mesh-like embodiment.

The exchange chamber is configured, very generally, to be substantially fitted within the outer catheter. This chamber is, in the preferred embodiment, fabricated from polyamide, or another polyester compound which is overall semi-permeable, but highly permeable to small molecular compounds such as N₂, O₂ or CO₂. This establishes the exo-pulmonary oxygenation (‘EXPO”), and the exchange chamber will be hereinafter known as the EXPO chamber.

In the preferred embodiment, the EXPO chamber is provided with a leading end, a central shaft, and a trailing end. The leading end is a blind pouch, and furthermore, the central shaft is provided with multiple corrugations which substantially increase the surface area. Multiple embodiments of such corrugations can be envisioned.

The EXPO chamber is provided with a leading end, a central shaft, and a trailing end. The leading end is a blind pouch, and furthermore, the central shaft is provided with multiple corrugations which substantially increase the surface area. Multiple embodiments of such corrugations can be envisioned regardless of the precise configuration of the EXPO chamber, this element is configured so that the chamber can be filled with gas. In one embodiment, the gas filling the chamber is a mixture which closely mirrors the gaseous admixture of the atmosphere at or near sea level. However, any ratio of gases, including 100% (“pure”) O₂.

The principal reason for providing the EXPO chamber with a large surface area relates to its function once filled with the gas mixture and positioned within the outer catheter sheath. It is presumed that the exchange chamber will be positioned within the outer catheter sheath after this sheath has been positioned within a vascular space such as the IVC or SVC. A large vein would be ideal because the velocity of the blood would be less than that of an artery, and the blood flow is constant rather than pulsatile. A large venous blood vessel would be optimal to expose the maximal amount of blood to gas exchange.

Once the EXPO chamber is filled with gas and positioned, the partial pressure of O₂, even in a normal (atmospheric) concentration would be approximately 160 mmHg (torr), which is actually slightly higher than that in alveoli - which is around 100 torr because of water vapor added as air entering during the respiratory process passes through the nasal and bronchial passages. In an extremely healthy individual volunteering as a study subject, the partial pressure of Oxygen in arterial blood, denoted as PaO₂, can exceed 100 torr when breathing room air.

Moreover, because the O₂ partial pressure is substantially lower in venous blood —PvO₂ — (so called “deoxygenated” blood), and in normal settings is around 40 torr, in a large vein, a very substantial gradient would be created that would drive molecular Oxygen (O₂) across the [semi-permeable] membrane and into the bloodstream. This is essentially why the lungs are so efficient in oxygenating the blood and would be yet another advantage to establishing the EXPO within a venous structure, although in moderate to severe ARDS, a major morbidity is decreased arterial 02 as well.

The blood flow in the larger venous structures such as the SVC or IVC is 10-35 cm / sec, so the uptake by RBC’s may not be complete on an instantaneous basis. However, transfer into the plasma will likely occur within 0.1 seconds, and therefrom, owing to the affinity of Hemoglobin for O₂, uptake by the RBC’s occurs within 1-2 seconds.

The interface between the [venous] blood and the external surface of the exchange chamber is enhanced by the porosity of the sheath catheter owing to the multiple fenestrations, or a mesh-like configuration.

The trailing end is coupled with the exchange valve of a unique, useful, novel and nonobvious apparatus, hereinafter known as the regulator, which governs the initial infusion of gas, and is responsible for exchanging the gas within the chamber as well as modulating the mixture of gas being infused into the chamber.

This device can be envisioned as having an intake valve, through which Oxygen and Nitrogen can be brought into the regulator. On/off valves control the flow of gas into a holding chamber, which is in turn connected to the exchange valve, which infuses gas into the exchange chamber of the EXPO.In the preferred embodiment, the intake valves for each of the gases (O₂, N₂) is actuated by a pressure sensor within the respective chambers within which of these gases are housed. The pressure sensors herein determine the amount of each chamber is filled with gas, based on partial pressures, and when low enough, actuate the intake valves to open and refill the chambers.

Furthermore, the separate Oxygen and Nitrogen Chambers are then connected via ducts to a central mixing chamber, which creates the gas mixture which will then be infused into the EXPO. This mixture can range from one similar to atmospheric/” room” air, essentially PN₂ of 600 and 02 160 up to 100% [pure] Oxygen, depending on the clinical circumstances. It is anticipated that controls will be provided to the regulator which allow clinicians to control this mixture.

The mixing chamber is then connected to the exchange valve, which, as mentioned above, which then directs the gas mixture into the exchange chamber of the EXPO.

It is anticipated that gas within the exchange chamber will require exchange from time to time. This relates to the presumption that O₂ uptake into the blood will ultimately deplete the O₂ within the exchange chamber, reducing the gradient and consequently the O₂ transfer will no longer be driven.

Beyond that, it is also anticipated that during the gas exchange, CO₂ will be driven into the exchange chamber owing to the steep gradient that exists. However, after a certain amount of time, this will increase within the chamber, thus reducing the gradient and limiting the exchange.

In the preferred embodiment, the gas exchange would be intermittent rather than continuous. Firstly, as this is how the lungs function, there may be some as of yet inexplicable benefit to organizing the gaseous exchange with the blood in that fashion.

Secondly, organizing a continuous flow of gases into and out of the exchange chamber would likely present technical difficulties, and require intricate channels within the exchange chamber to achieve this. Ergo, alternative embodiments to achieve this can be anticipated and are incorporated within the spirit and scope of this disclosure, they would present manufacturing and utilization challenges.

Perhaps a more critical consideration, however, mitigating in favor of intermittent gas exchange recognizes one potential complication of this procedure, that being the possibility of an interruption of the integrity of the inner with consequent release of gas directly into the bloodstream. This well-known pathology, which can occur through a variety of mechanisms, and is known as an “Air Embolism,” - literally a “clot” of air within the vascular system. Small air embolisms with volumes of less than 10 cc’s are usually insignificant and without clinical sequelae. However, larger volume events can lodge in major structures such as the Pulmonary Artery, leading to devastating complications such as Pulmonary Infarction.

In the preferred embodiment, the exchange valve will be bidirectional; on one side, a valvular component known as the evacuation valve opens, and suction removes the gases in the exchange chamber. This component then closes, and the second valvular component known as the fill valve opens, transferring the gas in the mixing chamber into the exchange chamber of the EXPO catheter.

It is anticipated that a pressure sensor is provided to the exchange valve, specifically to determine the pressure in the exchange chamber of the EXPO during the filling phase. At a certain predetermined pressure, which presumably can be varied by clinicians, the fill valve closes.

An important aspect of the pressure sensor is that if the integrity of the exchange chamber is breached, and gas passes directly through the wall and into the blood flow, filling the chamber would not result in an increase in pressure, which would alert clinicians in the form of an alarm. This alarm could be in various embodiments, such as a bell, oscillating sound or monotone sound, flashing light, or any other alarm known and acceptable to the art. Upon the indication by the alarm, clinicians would presumably discontinue use of the EXPO until the exchange chamber is removed and examined or preferentially replaced.

The clinical advantage, and problems solved by this system is that the exchange chamber would likely not have a volume which would create a clinically significant air embolism.

A continuous gas exchange, as mentioned above, is also conceivable and included within the spirit and scope of the invention. In such a system, a pressure sensor would also be utilized to govern the flow of gases, and to monitor the exchange chamber for any leaks of gases resulting from a disruption in the integrity of its wall.

### DETAILED DESCRIPTION OF THE DRAWINGS

The invention is best understood by studying the following detailed descriptions in conjunction with the context of the accompanying images, wherein like reference numbers refer to like structures, in accordance with common practice. Also, in accordance with common practice, the structures illustrated are not necessarily drawn to scale, nor can inferences of scale be developed with respect to such drawings. The embodiments presented and illustrations herein are general representations of the invention and are not nor can they be construed to be restrictive.

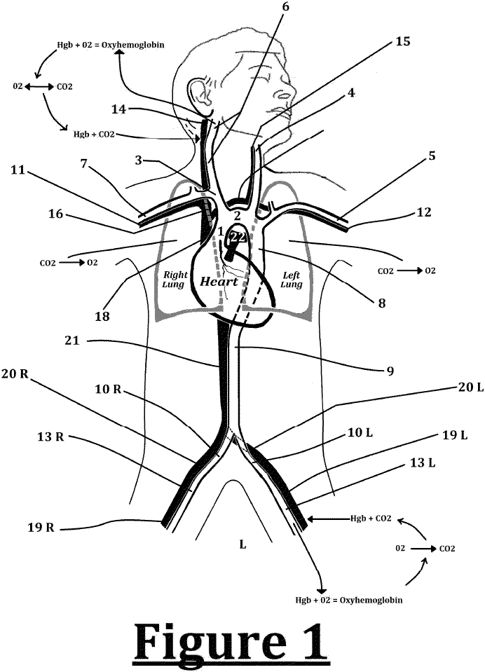

Therefore, turning attention to FIG. 1, the reader is provided with a critical overview of relevant cardiopulmonary anatomy and physiology. An understanding of these principles is fundamental to an understanding of the invention, and of its many objectives and problems it may solve.

This demonstrates a human subject in the supine position with the critical organs “Heart,” and “Lungs,” having been labeled, with the “Lungs,” outlined in gray to provide contrast. The circulatory system can be arbitrarily said to commence with the Ascending Aorta 1, which arises from the left ventricle of the heart and is transmitting the most “Oxygen-rich” blood - that with the highest Pa O₂ found anywhere in the peripheral circulation. This is because this blood has just returned from the lungs, where (as suggested by the denotation CO₂ --- O₂) at the alveoli / capillary interface, Carbon Dioxide is exchanged for Oxygen. This blood returns to the heart with very little of the O₂ having been taken up and utilized. From the Ascending Aorta 1, the blood moves into the Aortic Arch 2 which gives rise to three critical vessels: the Brachiocephalic artery 3, the Left Common Carotid artery 4, and the Left Subclavian artery 5. However, it is worth noting that the Ascending Aorta 1 gives off two fairly small but very important branches prior to the Arch, those being the left and right Coronary arteries, of which the left is illustrated as a small, unnumbered branch seen on the front of the heart. In this fashion the heart is actually fed by the highly oxygenated blood from the Ascending Aorta 1. With respect to this disclosure, the heart is also readily injured when ARDS prevents proper Oxygen exchange.

The Brachiocephalic artery 3 in turn divides into the right Common Carotid artery 6 and the right Subclavian artery 7.

The left and right Common Carotid arteries 4, 6 ultimately divide into external and internal Carotid arteries (not shown), with the external branches feeding the face and scalp and the internal branches providing the anterior cerebral circulation, irrigating the majority of the brain.

The left and right Subclavian arteries 5, 7 are the principal circulation to the upper extremities. These arteries can be seen closed accompanying the right and left Subclavian veins 11, 12, which in a corresponding fashion are the final common pathways for venous blood returning from the upper extremities.

After giving off the left Subclavian artery, the Arch is directed caudally and becomes the Descending Thoracic Aorta 8, which gives off a number of segmental branches in the thorax prior to passing through the diaphragm (not shown) and becoming the Abdominal Aorta, which gives rise to numerous branches which feed the kidneys and all the internal abdominal organs. Around the fourth lumbar vertebra, this artery divides into the left and right Common Iliac arteries 10 L, R which continue to feed the pelvic organs, and eventually become the left and right Femoral arteries 13 L, R.

The venous system carries blood back to the heart from all parts of the body. As demonstrated in the diagrams, and will be discussed further below, Oxygenated blood (PaO₂ > 100 torr) is carried to the tissues by the arteries and pass through capillaries where Oxygen is taken up by the tissues in exchange for Carbon Dioxide. The venous blood, relatively low in Oxygen (PaO₂ approx 40 torr) and significantly higher in CO₂, then returns to the heart where it is sent to the lungs for reoxygenation.

For example, the Carotid arteries irrigate the majority of the brain and in particular the expanded cerebral cortex. This blood is then collected by the cerebral venous system and ultimately carried to the right and left Internal Jugular veins 14, 15.

The right Internal Jugular vein 14 joins with the right subclavian vein 11 to form the right Brachiocephalic vein 16. This then continues toward the heart, joining with the left Brachiocephalic vein 17 which is formed by the junction of the left internal Jugular vein 15 and the left Subclavian vein 12.The junction of both Brachiocephalic veins results in the Superior Vena Cava 18 (“SVC”), which is one of the two large venous structures which empty into the right atrium of the heart.

In the lower extremities, in an analogous fashion, again is seen a series of vessels which gradually increase in size as they join with other veins, ultimately emptying in the heart. The left and right Femoral veins, 19 L and R, pass into the pelvis to become the left and right Iliac veins 20 L,R. These then merge into the Inferior Vena Cava (“IVC”), which is also one of the principal large veins draining into the heart.

FIG. 1 is also provided with two diagrams representing the arterial-capillary-vein circuit responsible for dispatching Oxygen into the tissues. Turning attention to the right Internal Carotid Artery 6, a curved arrow is seen leading from the artery to the equation “Hgb + O₂ = Oxyhemoglobin,” indicating that within an artery, Oxygen is being transported having reversibly bonded to Hemoglobin to create Oxyhemoglobin. This is the most efficient way of transporting Oxygen, and is especially efficient at the capillary level, which is represented by the statement O₂ CO₂ indicated by the second arrow. This statement represents the capillary level, wherein Oxygen molecules lose their affinity for Hemoglobin (owing to the low Oxygen pressure in peripheral tissues, in this case the brain tissues); O₂ is then released by Hemoglobin and dissolved into the tissues. CO₂ is higher in concentration in the tissues, and is exchanged into the blood, to be carried to the lungs.

This same exchange can be seen occurring in peripheral tissues, as represented by the same series of statements commencing with the left Femoral artery 13 L and culminating in the left Femoral vein 19 L.

A special circulation is the Pulmonary circulation. As has been stated above, the Superior Vena Cava/SVC and the Inferior Vena Cava IVC carry blood returning from all areas of the body (except the lungs), with these vessels passing into the right atrium. As the heart pumps, the blood is pumped from the atrium through the Tricuspid valve and into the right ventricle. The Pulmonary artery 22 emerges from the right ventricle, carrying the absolutely Oxygen poorest blood of any vessel (as opposed to all other arteries, which carry Oxygen rich blood coming from the heart).

The blood then goes into the lungs, where it passes through the capillaries which are intimately related to the alveoli. That interface results in the discharge of CO₂ from the blood into the alveoli, ultimately to be exhaled. Simultaneous with the transit of CO₂, O₂ which is at a higher concentration within the alveoli, passes into the blood, thus “reoxygenating,” it. This is represented by the statement “CO₂---O₂ occurring in the lungs.

At that point the “Most Oxygen,” rich blood enters the left atrium through the pulmonary veins, which are generally 4 in number, but may vary. From the left atrium, the blood is pumped through the mitral valve and into the left ventricle, and thence from into the Ascending Aorta 1. This review is more than academic. It is fundamental to recognizing the many objectives of the invention, and the anatomy is critical to understanding where the EXPO catheter would be positioned for maximal effectiveness, as well as marginalizing the potential for complications.

In the most ideal placement, the EXPO would be positioned in a large venous structure near the heart. A large vessel would increase the efficiency of the invention by exposing a larger volume of blood to the exchange chamber containing the Oxygen rich environment. The velocity of the blood flow in slower in the venous system than the arterial system, and the pulsatile nature of the arterial flow suggests that during systole, the compression cycle of the heart, blood would move so quickly that the chances for gas exchange would be substantially compromised.

In the preferred placement, the catheter would be positioned through the Femoral Veins 19 R, L and ultimately into the Inferior Vena Cava / IVC 21. Alternatively, this could be disposed through the one of the Jugular veins 14, 15 and ultimately into the Superior Vena Cava / SVC 18. This would expose the high content of Oxygen within the exchange chamber to the Oxygen-depleted environment of one of these vessels. The device could also be disposed through the left or right Subclavian veins 11, 12 and again into the SVC 18. Obviously, other strategies could be anticipated and executed. For reasons which have already been elucidated, an arterial placement would appear to be significantly less advantageous.

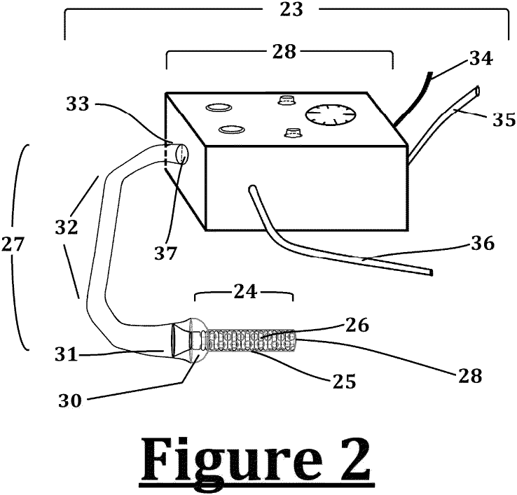

The device 23 is exhibited in its entirety in FIG. 2. In this elevated schematic view of the isolated system 23, one notes the catheter 24 representing the combined outer sheath 25, which for contrast and the multi-corrugated exchange chamber 26. To achieve better contrast, the outer sheath 25 has been illustrated in gray. The trailing end 30 of the catheter is reversibly coupled with the leading end 31 of the connection tubing 27, thus facilitating the transfer of gases to and from the Regulator 28.

The input of gases into the Regulator 28 is conducted via the Oxygen input 34 and the Nitrogen input 35. As will be discussed further below, the gases will be combined into specific admixtures as directed by the clinicians managing the patient. After this admixture is created within the Regulator 28, it is then delivered via the connection tubing 27. The trailing end 33 of the connection tubing 27 is reversibly coupled to the Exchange valve 37 of the Regulator 28. This valve 37 is bidirectional insofar that it is provided with both an outflow valve and a return valve. For clarity purposes, these valves are not illustrated in this image, but will be illustrated and discussed in additional images below. At the beginning of a cycle of function of the EXPO system, the gas within the exchange chamber 26 is first removed through the connection tubing 27 to the return valve that the Exchange valve 37 has been provided with, which accepts the gas to be removed. This gas is then delivered out of the Regulator 28 through the Discharge port 36. The Exchange valve 37 is also provided with an outflow valve, and upon removal of the gas from the connection tubing 27 and the exchange chamber 26, the gas exchange is completed by infusion of the admixture of gas from the Regulator 28 through the outflow valve of the Exchange valve 37. It is noted in FIG. 2 that the trailing end of the connection tubing 27 is reversibly coupled with the Exchange valve 37; the configuration of this valve is such that a bidirectional flow of gases can be created. This is facilitated by the configuration of the valve, which can rotatably change the direction of the gas flow to either force the admixture of gas into the connection tubing 27, and hence into the trailing end 30 of the EXPO catheter 24 or can be rotated to suction the gas out of the connection tubing 27 and the catheter 24, to be discharged through the Discharge port 36. The manner by which this unique, useful, novel and nonobvious system provides its benefits by the exchange of gases within the exchange chamber 26 resulting from the bidirectional flow of gases as dictated by the design of the connection tubing 27 and its transfer of gases from the Regulator 28 to the exchange chamber 26 will be further elucidated in greater detail in additional images below.

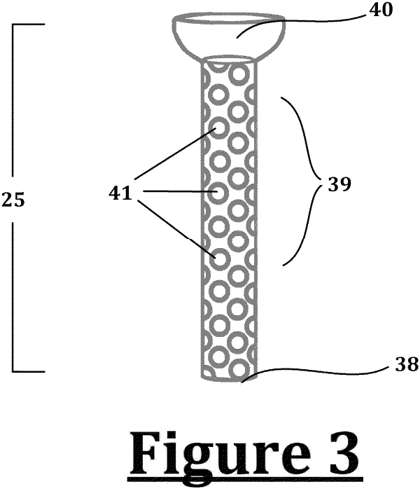

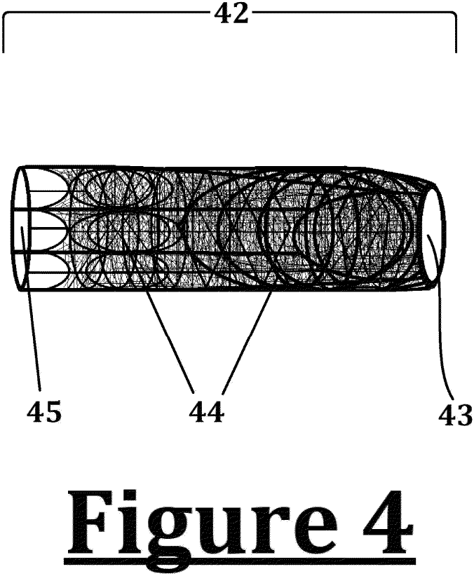

A top view of the outer sheath 25 of the catheter is seen in FIG. 3. This is provided with a leading end 38, a shaft 39, and a trailing end 40, and is substantially a cylinder with a central channel and an open leading 38 and trailing end 40 so that it can be disposed over a needle which is used to initially introduce the EXPO catheter into a vascular structure. It is anticipated that the outer sheath would be fabricated from Teflon, polyurethane or any of the polyesters known and acceptable to the art. In the preferred embodiment, or any alternative embodiment, it is anticipated that the outer sheath (as well as the outer surface of the exchange chamber) will be coated with Heparin, or some other thrombolytic agent.

The shaft 39 is, in the preferred embodiment, configured to have multiple apertures 41 which in turn expose the flowing blood directly to the outer surface of the exchange chamber, which is critical in encouraging the exchange of gases. Multiple alternative embodiments of the outer sheath can be anticipated, one of which is illustrated in FIG. 4 below. Other embodiments can be envisioned by those familiar with the art, and all such embodiments are incorporated within the spirit and scope of the invention.

The trailing end 40 of the outer sheath is provided with a general configuration which allows reversible coupling of the sheath, and in turn the catheter, to the leading end of the connection tubing. One embodiment of such a configuration is demonstrated in FIG. 3, but any embodiment which can be envisioned is incorporated within the spirit and scope of this specification.

One alternative embodiment is shown in the lateral view in FIG. 4, in which the outer sheath 42 is again fabricated from any substance known and acceptable to the art and is configured to represent a fine mesh surrounding the exchange chamber. The leading end 43 is again an aperture leading into a central channel, into which the exchange chamber is positioned. The shaft 44 is seen as composed of the mesh, and the trailing end 45 is again configured to reversibly couple with the leading end of the connection tubing.

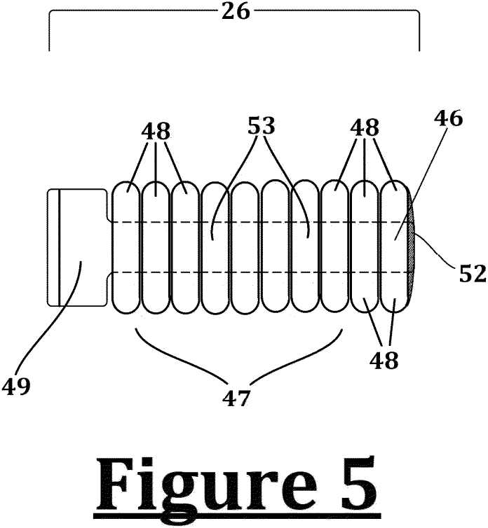

The exchange chamber 26, as shown in the lateral perspective in FIG. 5, is configured to be inserted into the outer sheath so as to allow the circulating blood to pass through the apertures or mesh constituting the outer sheath, thus facilitating direct contact between the RBC’s and the outer surface of the exchange chamber. It is anticipated that the exchange chamber will be fabricated from polyamide, or any other polyester or other substance acceptable to the art. The exchange chamber is responsible for the critical gas exchange which characterizes the unique, useful, novel and nonobvious aspects of this invention.

The exchange chamber is also provided with a leading end 46, a shaft 47 and a trailing end 49. Several critical aspects of the exchange chamber are to be noted.

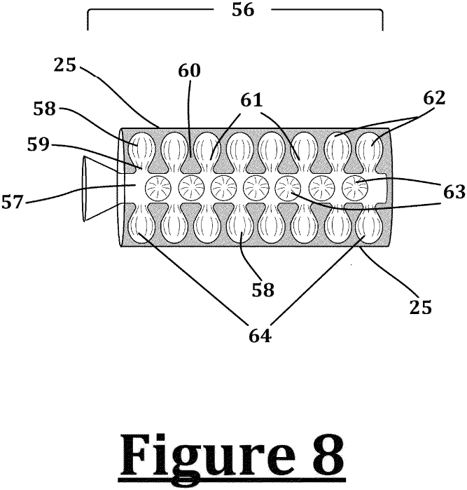

In particular, the multiple corrugations 48 of the exchange chamber create a large surface area for gas exchange between the admixture of gases within the exchange chamber and the RBC’s in the blood flowing along the outer surface of the exchange chamber. The large surface area is critical to accomplish the many goals and objectives of the invention. Multiple embodiments of the of the exchange chamber can be anticipated, some of which are illustrated in FIGS. 8-10, and in fact, more alternative embodiments of the exchange chamber can be envisioned than any other component of the EXPO system, all of which are incorporated within the spirit and scope of the invention.

The leading end is provided with a cap 52, displayed as the hatched area, which is secured against the aperture at the leading end of the outer sheath. This feature may not be seen in all embodiments, and embodiments lacking the feature are anticipated and could be envisioned by those familiar with the art. A possible advantage of such a cap could be to minimally retard blood flow through the space between the outer sheath and the exchange chamber thus containing blood within that space for a slightly longer period of time, and hence potentially increase the gas exchange occurring therein.

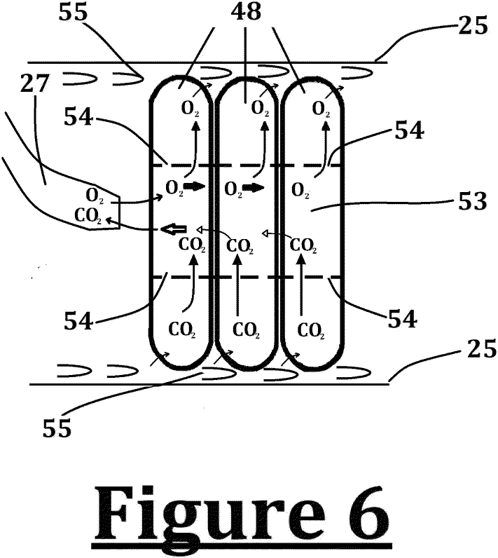

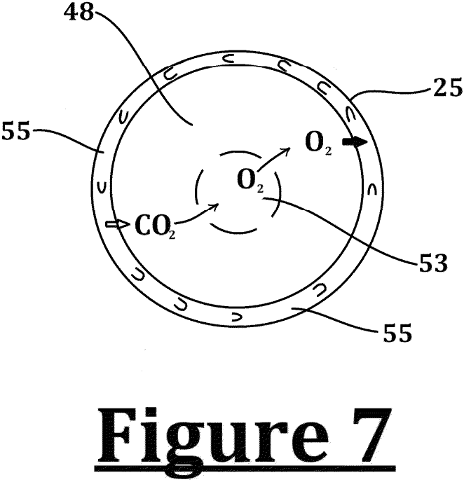

The central shaft is provided with a central gas delivery channel 53, which circulates the gas admixture throughout all of the sub-chambers which are created by the corrugations and / or other multiple surfaces provided to the multiple embodiments of the exchange chamber. It is proposed that the desired gas admixture is delivered into this gas delivery channel, which then communicates to the chambers created by the corrugations, thus promoting gas flow therein. This mechanism is more completely illustrated in FIGS. 6 and 7 below.

The trailing end 49 of the exchange chamber 26 is configured to reversibly couple with the leading end of the connection tubing. Thus, from the Exchange valve through the exchange chamber, a closed circuit for gas exchange is maintained.

The exchange of gases is schematically illustrated in FIG. 6, which is a lateral perspective of several exemplary corrugated segments 48 previously shown (also in the lateral view) in FIG. 5. These segments are multiple sub-chambers of the exchange chamber, thus creating a significantly greater surface area, ergo thereby creating a large gas exchange surface. The central gas delivery channel 53 is positioned centrally within the corrugated segments 48, with these segments positioned circumferentially around the central delivery channel 53; this relationship is also demonstrated in the transaxial view seen in FIG. 7. Multiple apertures 54 provided to the central channel 53 creating a free communication between the [sub-chambers] of the corrugated segments 48 and the channel 53. The outer sheath 25 is seen above and below the exemplary segments 48 shown herein, representing the anticipated configuration by which the outer sheath 25 surrounds the segments 48. This configuration, as previously reviewed, anticipates blood flow 55 to be present between the outer sheath and the outer surface of the exchange chamber; this is represented by the hemi-ovals, characterizing that blood flow is laminar in nature.

The gas admixture is delivered from the Regulator through the connection tubing 27, which is also schematically represented herein, to the exchange chamber, as demonstrated in FIGS. 2 and 5. The admixture being delivered has a higher concentration of Oxygen, as indicated by the upper series of arrows and the [solid] arrow showing Oxygen (O₂) flowing into the central gas delivery channel 53 from the connection tubing 27. O₂ flows therefrom through the apertures 54 in the walls of the central channel 53 into the corrugated segments 48, represented by the upper set of large curved arrows; from there, the Oxygen (O₂) is seen diffusing through the outer surfaces of the corrugations 48, including the spaces between them, and into the blood flowing past 55; this gas exchange is represented by the upper set of small curved arrows. Presumably, Oxygen will pass from an area of higher concentration to a lower concentration, resulting in combination with hemoglobin forming oxyhemoglobin; gas exchange therewith is hence promoted.

The higher concentration of Carbon Dioxide is in the blood, relative to the admixture, and hence as schematically represented, CO₂ will be driven from the blood into the exchange chamber, as represented by the lower set of small curved arrows. Ultimately, as shown by the lower set of larger curved arrows, it passes through the apertures 54 and into the central channel 53 and thence from, as represented by the [open] arrow, removed through the connection tubing 27. Although the exchange of gases is schematically represented with the Oxygen exchange on the top of the page and the Carbon Dioxide exchange on the bottom, it is to be understood by the reader that such exchanges occur simultaneously along all of the surfaces of the exchange chamber.

This system is further understood by reviewing a transaxial view of the exchange chamber 26 in FIG. 7. Again, it is seen that the corrugated segments 48 are positioned circumferentially around the central gas delivery channel 53.

This configuration, in turn, is represented as positioned within the outer sheath 25. Again, blood flow 55 is seen between the two membranes. The gas exchange is seen occurring between the admixture in the corrugated segments 48 and the lumen 55 created by the potential space between the corrugated segment 48 and the outer sheath 25. As in FIG. 6, this lumen is represented by the series of hemi-ovals, characterizing that blood flow is laminar in nature. O₂ is seen being driven from the higher concentration within the corrugated segments 48 into the blood flowing within the lumen 55, with its lower concentration.

Furthermore, the CO₂ is in a significantly higher concentration in the blood 55 with respect to the corrugated segments 48, so it is easily driven into the segments 48. Again, when the gas in the exchange chamber is exchanged, this is presumably removedAs previously discussed, the exchange chamber presents the potential for a large number of alternative embodiments. One such embodiment 56 is presented in FIG. 8, in which an exchange chamber 56 is again provided with a central gas delivery channel 57 which presents the gas admixture to a series of baffles 58, which are segmented similar to the corrugated segments in FIGS. 2, 5, and 6. However, these baffles 58 are substantially spherical in configuration, arising from the central delivery channel 57 via a narrow isthmus 59. This configuration will create a large surface area favoring an increase in gas exchange between the interiors 61 of the baffles and the blood flowing in the lumen 60 (shown in light gray for contrast) between the outer sheath 25 and the interior of the baffles 61. It can be noted that these baffles 58 are configured such that a row 62 of them arises from the superior aspect of the central channel 57 whilst another row 63 arises 180° opposite, and another row 64 arises from the central channel 57 and is positioned intermittently between the superior 62 and inferior rows 63, configured to be extending laterally (end-on, as seen by the reader). Positioning the baffles in this fashion will increase the surface area responsible for the gas exchange, which is one of the principal problems this invention seeks to solve.

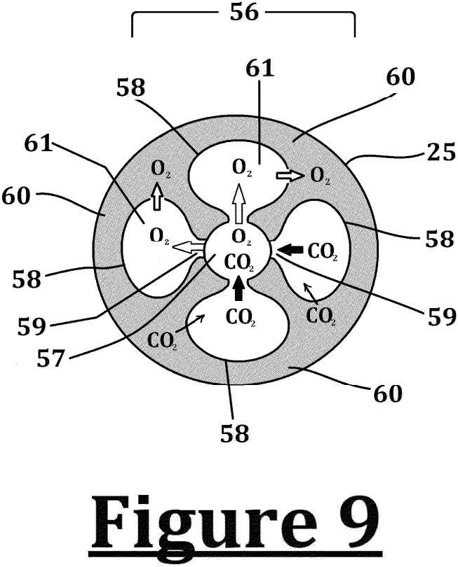

This embodiment is further elucidated in FIG. 9, in which the embodiment 56 in FIG. 8 is now seen in a transaxial perspective. In this drawing, the outer sheath 25 again is seen creating a potential space or lumen 60 (demonstrated in this image as light gray) between the outer sheath 25 and the exchange chamber 56. When reduced to practice, it is anticipated that the blood will be flowing through the lumen 60, thus creating the environment for gas exchange between the interior 61 of the baffles 58. One notes, as described in FIG. 8, that a row of baffles 58 are positioned arising from the superior aspect of the central gas delivery channel 57, and another row arises from the inferior aspect. It can also be seen that these two rows of baffles 58 are interspaced with rows of baffles 58 that arise orthogonal to the orientation of these two rows.

The illustration reveals that Oxygen O₂ is delivered via the central gas delivery channel 57, which passes through a narrow isthmus 59 into the interior 61 of the baffles 58. As illustrated schematically, the O₂ then passes through the permeable membrane that comprises the exchange chamber 56, into the blood in the lumen 60 where it combines with Hemoglobin.

Further illustrated schematically is the route of Carbon Dioxide CO₂ which passes from the higher concentration in the blood in the lumen 60 and into the interior 61 of the baffles 58, and from thence into the central gas delivery channel to be removed. Obviously, although the gas exchange is separated in the illustration between the Oxygen exchange, shown at the top of the image, and the Carbon Dioxide exchange at the top of the image, it is recognized that such exchanges occur simultaneously throughout the entire construct.

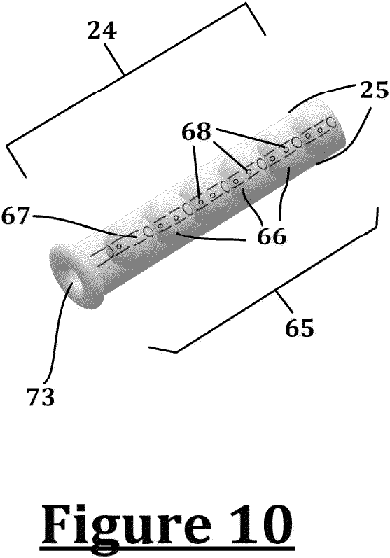

Another alternative embodiment of the exchange chamber is seen in the lateral elevational perspective in FIG. 10. Here, the catheter 24, is again comprised of an outer sheath 25 is again surrounding the alternative embodiment 65 of the exchange chamber, thus creating a lumen 69 through which the blood flows thus providing a venue for the gas exchange. The connection tubing (not shown) couples with the trailing end 73 of the central gas delivery channel 67, with a sequence of multiple larger spherical baffles 66 positioned along the delivery channel 67, noted by the interrupted lines and larger solid circles; a series of apertures 68 communicate the baffles 66 with the central gas delivery channel 67. The Oxygen is delivered through the central channel 67 and then through the apertures 68 into the baffles 66 for exchange with the blood. As previously disclosed, the Carbon Dioxide is then driven in the opposite direction.

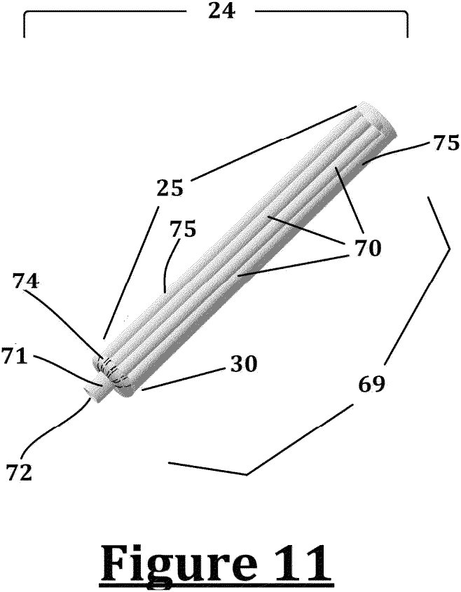

Still another embodiment of the exchange chamber 69 that can be anticipated is provided with multiple longitudinal chambers 70 which are circumferential to and continuous with central delivery channel 71. This is seen in an elevational perspective in FIG. 11. As in all other embodiments, this unique, useful, novel and nonobvious alternative embodiment of the exchange chamber 69 is positioned within the outer sheath 25, thus comprising the catheter 24. The trailing end 72 of the central delivery channel 71 is configured to couple with the connecting tubing (not shown) thus delivering the Oxygen-rich gas mixture and serving as the portal to remove the mixture when the Carbon-Dioxide partial pressure has increased. The trailing end 72 of the central delivery channel 71 is provided with a manifold 74, as illustrated by the interrupted lines, this manifold 74 thereby delivering gas between the central gas delivery channel 71 and multiple longitudinal chambers 70 which are positioned circumferentially around the central delivery channel 71. The exterior surfaces of these chambers are then in direct contact with the blood in the lumen of the catheter, thus facilitating the gas exchange.

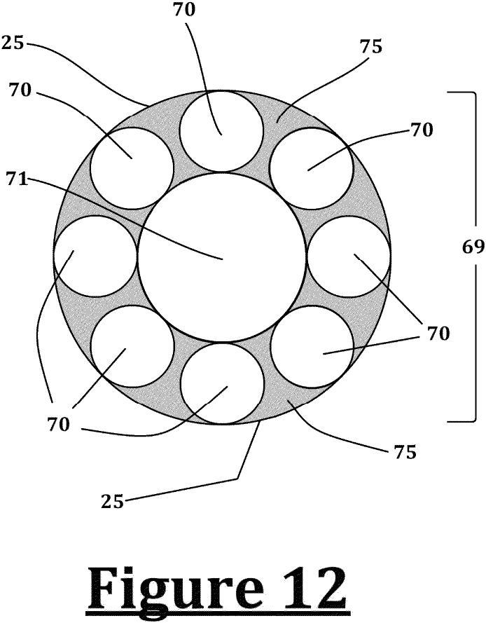

The locus of the gas exchange/transfer may be better appreciated in the transaxial view of this embodiment 69 shown in FIG. 12, as the lumen 75 (shown in light gray for contrast) created by the space between the outer sheath 25 and the longitudinal chambers 70 is readily noted; it is also noted that the lumen 75 (gray) extends in between the longitudinal chambers 70. Here, it can be seen that the extended surface areas of the multiple longitudinal chambers 70, which are positioned circumferentially around the central delivery channel 71, serve as the primary sites for the exchange with the blood circulating in the lumen 75.

Other embodiments can also be anticipated; all such embodiments of this unique, useful, novel, and nonobvious that can be envisioned are included within the spirit and scope of the invention.

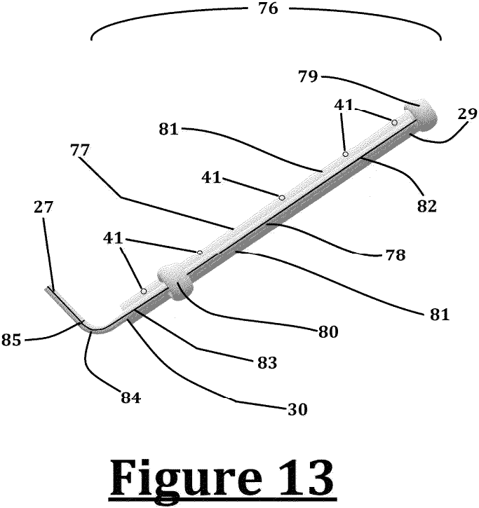

One such embodiment increases the gas exchange by intermittently interrupting the blood flow through the target vessel, thus providing a greater time period for gas exchange to occur with the blood within the lumen of the catheter. This is accomplished by positioning inflatable balloons at the leading end of the catheter as well as at the point where the catheter is passed into the vessel. This is demonstrated in FIG. 13, wherein an alternative embodiment of the catheter 76 shows that the outer sheath 77 is provided with the anticipated apertures 41 (shown in a limited fashion for clarity purposes), into which an embodiment of the exchange chamber 78 has been placed; any of the exchange chamber embodiments which have been disclosed thus far could be used in this unique, useful, novel, and nonobvious embodiment. Alternatively, any embodiments envisioned by those familiar with the art may be also used. FIG. 13 displays a simple embodiment of a straight exchange chamber 78 to underscore that any configuration of the exchange chamber is acceptable in this embodiment. Furthermore, the exchange chamber is seen superimposed as a “ghost,” image, to represent its position within the center of the outer sheath 77.

The unique aspect of this embodiment relates to two unique components of the outer sheath. A deployable balloon 79 is provided to the leading end 29 of the catheter, specifically the outer sheath 77, with a second such balloon 80 being provided to the trailing end 30 outer sheath 77 at a point slightly proximal to the entry point into the target vessel. The balloons are demonstrated in their fully deployed configuration in the drawing; it is understood that when fully deflated these are incorporated within the outer sheath and add little to the overall profile. The balloons 79, 80 can be inflated manually by syringes (not shown); alternatively, they can be inflated by an automatic system which can be programmed to inflate them at regular intervals. Regardless of the actuating mechanism, inflation is achieved by the passage of air or fluid from an inflation syringe or reservoir, the air or fluid then being transferred through small tubes 82, 83 which pass along the external surface of the connecting tubing 27 until the junction 85 with the trailing end 84 of the exchange chamber 78, and then being incorporated into the outer sheath. Ultimately, the tubing 82, 83 couples with the balloons 79, 80 respectively.

The balloons 79, 80 are also incorporated within the outer sheath 77 such that upon inflation, as pictured, each balloon expands outward to press against the wall of the blood vessel into which the catheter 76 has been positioned; additionally, the balloon 80 at the trailing end of the catheter 76 expands inward and is brought up against the outer surface of the exchange chamber 78. The balloon 79 at the leading end 29 of the catheter 76 expands to completely occlude blood flow through the vessel, and in this fashion, the blood flow in the segment of the vessel occupied by the catheter 76 is discontinued or “trapped.” This is maintained as such for a limited period of time (e.g., 5-10 seconds), and as such, the non-flowing blood within the lumen 81 of the catheter 76 has a longer period for the gas exchange, resulting in greater gas exchange, which is the object of this embodiment 76.

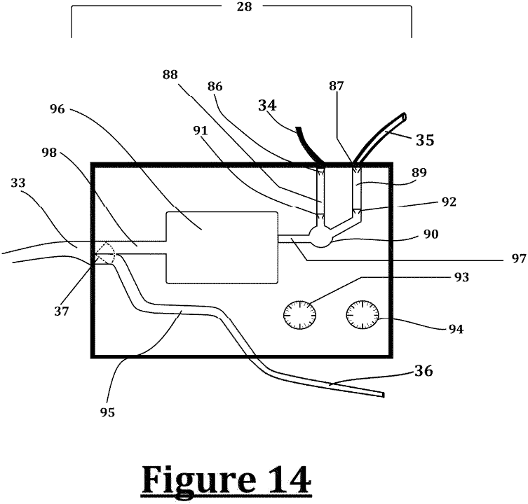

The structure and function of the Regulator 28 is discussed in greater detail in FIG. 14. The schematic representation of this unique, useful, novel, and nonobvious device 28 is understood to represent merely one potential schematic of this embodiment, and it is fully understood that those familiar with the art can envision multiple other schematics that would achieve the many obj ectives of this device. The reader should note that the Regulator 28 would, in the preferred embodiment, likely be a box or similar configuration, and the structures illustrated would therefore be contained within such a box. Although these structures are not illustrated with interrupted or “dotted,” lines, which has been the convention used throughout this application to illustrate internal structures, they nevertheless would be internalized structures.

This component (Regulator 28) of the invention disclosed would be the most subject to variations, and the preferred embodiment is but one of many configurations of this device which could been visioned by those familiar with the art, but all such configurations would be included and embodied within the spirit and scope of the invention.

The principal objective of the Regulator 28 is to provide the physician and respiratory staff with a mechanism for directing and controlling the partial pressures of the gases which are presented to the exchange chamber for gas exchange with the blood.

Whilst it would, on the surface of any review, appear logical to present 100% (pp 760 ^{torr}) to the exchange chamber for exchange, in reality this may well not be the case, and in fact, doing so may result in a Oxygen partial pressure in the blood of several hundred, which has been definitively shown to have a deleterious effect in some settings and likely has a negative effect in a number of other settings.

Therefore, the data strongly suggests that a patient with ARDS will benefit by having the partial pressure of O₂ at or slightly above normal, but not massively elevated. Very high levels of Oxygen can be associated with increased levels of free radicals, and at least on a theoretical basis lead to increased tissue damage.

The Regulator 28 achieves this objective by receiving O₂ from an external input tubing for O₂ 34, which, in turn, passes through an intake valve 86 and from there, is disposed through the Oxygen transfer tubing 88 to the mixing chamber 90. This transfer of gases is governed by an Oxygen tubing valve 91, which in turn is under the control of an Oxygen control dial 93. This allows the physician or respiratory therapist to control the amount of Oxygen, which is introduced into the mixing chamber 90, which will ultimately be the O₂ concentration in the gas mixture which will be delivered to the exchange chamber of the catheter.

In an analogous position, Nitrogen (the other principal atmospheric gas which is normally exchanged during respiration), is delivered through an N₂ input tubing 35. The gas is then disposed through a Nitrogen intake valve 87 and into a Nitrogen transfer tubing 89. Transfer of N₂ from here is governed by the Nitrogen tubing valve 92, which is under control of the Nitrogen Control dial 94. In a similar fashion, this controls the concentration of Nitrogen being introduced into mixing chamber 90, which again is reflective of the gas concentration being introduced into the exchange chamber of the catheter.

Once the anticipated composition is within mixing chamber 90, it is disposed through the combined transfer tubing 97, and into the input reservoir 96. The reservoir 96 can be actuated by one of several mechanisms, including bellows that can be actuated, or by creating pressure gradients, and other mechanisms such as a pressure chamber; as such, no particular mechanism is demonstrated herein, but rather the generic image of the reservoir 96.

From the reservoir 96, the gas mixture is transferred through the reservoir output tubing 98, which is controlled by the Regulator Exchange Valve 37, which controls the direction of flow of gas out of and back into the Regulator 28. In the first instance, the gas flows out of the reservoir output tubing 98, past the valve 37, and into the trailing end of the connecting tubing 33. As previously reviewed in FIG. 2, the gas is then disposed through the connecting tubing 33 and into the exchange chamber of the catheter (not shown). After a specified time, the gas within the chamber is exchanged by removing the gas within the exchange chamber, which is carried back to the Regulator 28 again by the connecting tubing 33.

At the entry point into the Regulator 28, the Regulator Exchange Valve 37 will actuate to direct the gas re-entering the Regulator 28 into the Exiting gas conduit 95. This directs the gas exiting the catheter, presumably with a lower concentration of O₂ and CO₂ (which would not be found in the mixture being infused into the exchange chamber), into the Discharge port 36, which disposes of the gas removed from the Regulator 28.

Other components of the Regulator can be anticipated and envisioned. These could include a timing device to actuate the gas exchange at regular, predetermined intervals, as well as a sensor to interpret the partial pressure of CO₂ of gas exiting and adjust the time interval of the exchange; accordingly, this is further elucidated in schematic fashion in FIG. 17 below.

One can also imagine that there could well be other arrangements in terms of gas tubings entering/exiting the Regulator 28. Other modifications can also be envisioned by those acquainted by the art, all of which would be incorporated within the spirit and scope of this application.

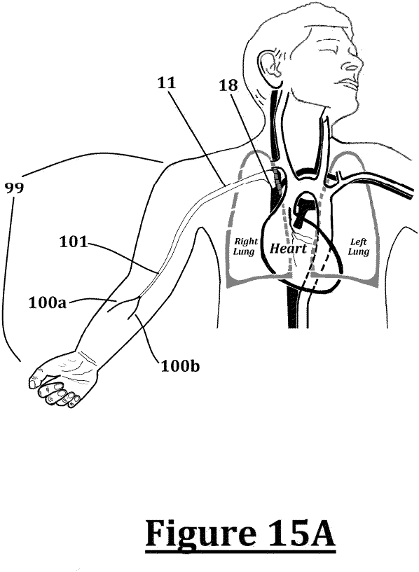

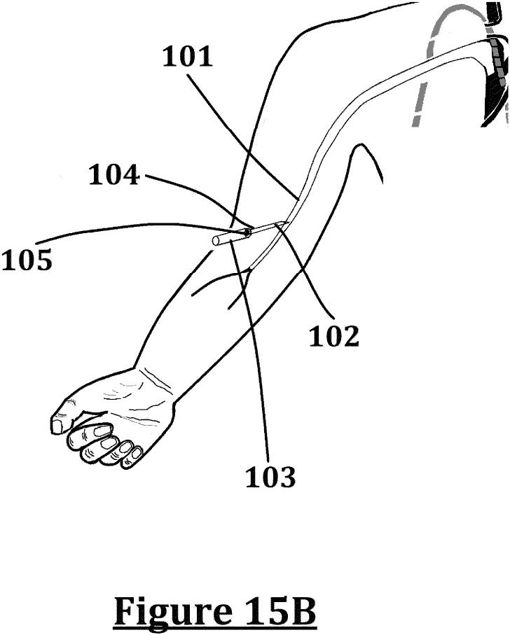

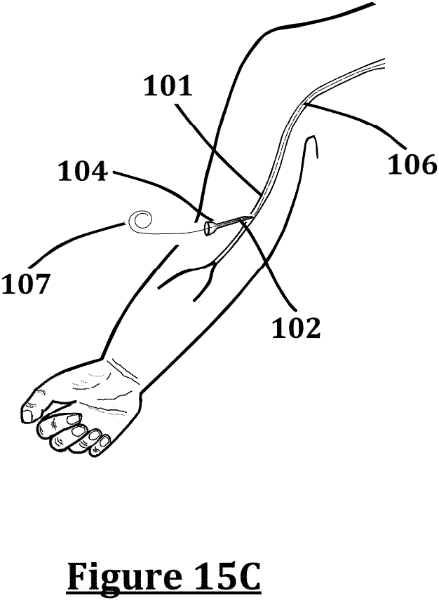

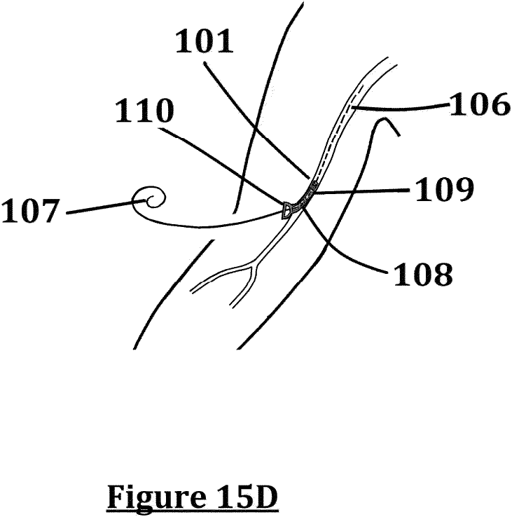

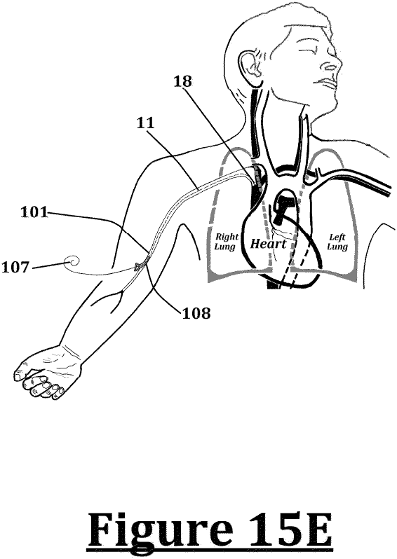

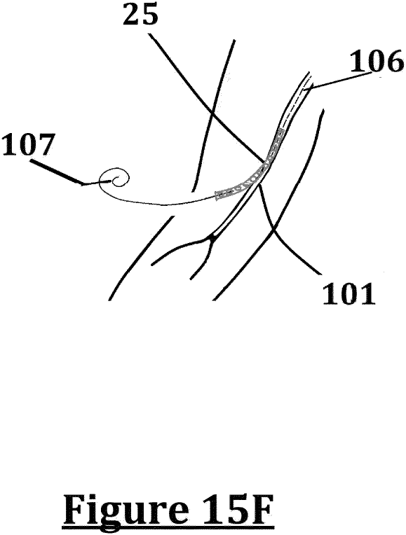

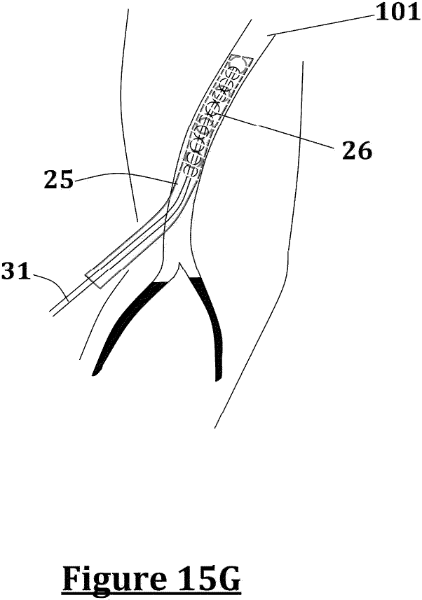

The preferred method of inserting the EXPO Catheter disclosed herein would utilize standard methodology for insertion of such a device within a vascular space. This is illustrated in FIG. 15A, in which the right arm is selected as the insertion site with the ultimate goal of passing the catheter into the right subclavian vein and ultimately into the Superior Vena Cava.

In FIG. 15A, a view of the anterior aspect of the upper torso and head, the right arm is portrayed in the volar position. Two smaller veins 100a/100b are seen coming together to form a large antecubital vein 101, which shall be used as the venous access site in these exemplary illustrations, bearing in mind that multiple other sites could be approached in a similar fashion.

It should be noted that in FIG. 15A, the right subclavian artery and its branches have been removed, and the right subclavian vein and its tributaries are portrayed in outline similar to arteries elsewhere (in contrast to FIG. 1).

In FIG. 15B, which for the purposes of clarity is an enlarged view of the right arm focused on the antecubital fossa, the first step is shown in which an introducing needle 102 accesses the antecubital vein 101. Good position within the vein is confirmed by flashback of blood 105 into a syringe 103 which is reversibly coupled to the trailing end 104 of the syringe 103. This assures good position within the target blood vessel 101.

FIG. 15C reveals that upon positioning the needle 102 as demonstrated by the flashback, the syringe is removed and a guide wire 106 is disposed through the trailing end 104 of the introducing needle 102 and into the right antecubital vein 101. The guide wire 106 is represented as an interrupted line as it passes through the lumen of the needle and transgresses the endovascular space. The trailing end 107 of the guide wire 106 must be maintained externally and cannot be released, even very transiently, as this would pose the threat of being carried intravascularly without the opportunity of retrieval. It is common to maintain the trailing end of such guide wires on a dispenser or similar configuration, which reduces the chances of unintentional intravascular displacement of the entire wire. Although such an embodiment is not illustrated, it is, of course incorporated within the spirit and scope of this invention.

As will be reviewed further below, there are multiple sites within the vascular system which are appropriate for placement of the catheter. In this instance, insertion into the antecubital vein could result in placement within the antecubital vein, or transvascular positioning within a site more proximal in the venous system such as the Subclavian Vein or even the Superior Vena Cava.

In an alternative embodiment which is illustrated in FIG. 15D and is frequently utilized in such art, after the introducing needle penetrates the target blood vessel, there is a soft polyester introducer 108 which is positioned around the external surface of the needle prior to venipuncture. Upon successful vascular entry, the introducer 108 is then disposed over the needle and into the blood vessel 101, withdrawing the needle simultaneously. The guide wire 106 is then disposed through the trailing end 110 of the introducer 108, and into the target vessel 101. This is done because manipulating the wire 106 through such an introducer 108 is achieved more readily, and with far less potential for damage to the wire 106, particularly at the point where the wire 106 passes through the leading end 109 of the introducer 108. When this maneuver is performed through a needle, turbulence of the flowing blood can result in shearing of the wire by the sharp leading end of the needle, a complication which has been reported.

In yet another alternative method – recognized but not shown herein – upon positioning the introducer and removing the needle, a cap at the trailing end of the introducer is temporarily closed, thus preventing egress of blood through the trailing end. When the outer sheath is ready to be positioned directly through the introducer, the cap is opened, and the outer sheath is disposed through the introducer without the use of a guide wire.

It is also noted that as the guide wires are almost always fabricated from metal, they are typically radiopaque, so many practitioners perform placement of this type of device under fluoroscopic guidance. Whilst this would not be the preferred method, it is certainly incorporated within the spirit and scope of this application.

Regardless of the technique employed, the ultimate goal is to have the guide wire 106 seated within a target vessel, as illustrated in FIG. 15E. This will then permit the catheter to be threaded into the optimal intravascular position. In this drawing, one again notes the trailing end 107 of the guide wire 106 which has been disposed through the introducer 108, with the guide wire 106 being threaded through the right antecubital vein 101 into the right subclavian vein 11, from which it passes into the Superior Vena Cava 18, which may be one of the preferred positions for the catheter.

In FIG. 15F, an enlarged view of the introduction of the invention into the vascular system, the outer sheath 25 [of the catheter] can be seen as it is disposed over the guide wire 106 into the anticipated intravascular position. In this image, the sheath 25 is being disposed into the [right] antecubital vein 101, comporting with the previous images in this sequence. As previously disclosed, the trailing end 107 of the guide wire 106 must be maintained under control outside the corpus of the patient.

As the next and final step, also shown as an enlarged view portrayed in FIG. 15G, the guide wire has been withdrawn and the exchange chamber 26 is now positioned within the outer sheath 25, thus comprising the Catheter 24 per se inside of the target vessel 101. The leading end 31 of the connecting tubing is seen coupling with the exchange chamber 26 such that the invention is now positioned to be deployed. It is of course understood that the embodiments and in particular those of the inner and outer chamber are merely representative of specific embodiments which have been already disclosed above. Any conceivable combination of exchange chamber 26 embodiments with outer sheath 25 embodiments herein disclosed are included within the spirit and scope of this invention. Furthermore, it is anticipated that those familiar with the art may posit, postulate or propose embodiments of the elements of this invention which are distinct from those disclosed above; such embodiments, when applied to achieve the distinct objectives and goals of this invention, are clearly included within the spirit and scope of this invention.

In this unique, useful, novel and nonobvious disclosure, it can be anticipated that various sizes of the catheter 24 comprised of the exchange chamber 26 and an accompanying outer sheath 25 are disclosed and provided. These sizes are, of course, correlated with the size of the target vessel as well as the entry point through which they can be introduced.

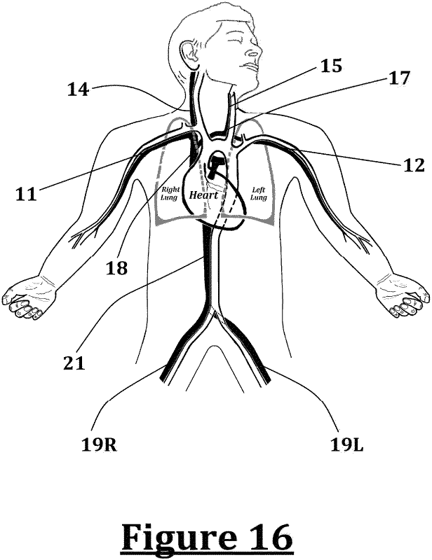

Multiple sites throughout the body are suitable for placement of the EXPO catheter, as recognized in FIG. 16, wherein some of the principal sites are identified. It is anticipated that to this end, the venous system would be advantageous as compared to the arterial system, primarily because the speed of the blood flow is considerably more rapid through the latter. Considering the fundamental manner by which the EXPO works, it can be anticipated that the device will function more effectively and result in more richly oxygenated blood with slower blood flow. It can even be recalled that an alternative embodiment disclosed above (See FIG. 13) utilizes intermittent cessation of the blood within the target blood vessel to increase the oxygenation of the blood therein.

Pursuant of that thesis, large venous structures, in which a very significant amount of blood is flowing at a fairly slow rate become the most attractive sites for positioning of the EXPO catheter. As identified in FIG. 16, the Superior (SVC) and Inferior Vena Cava 18, 21 are [obviously] the most attractive sites within which to position the EXPO catheter, although these structures are both central structures and therefore not directly accessible.

Further scrutiny of the drawing, however, reveals that the most desirable position may be within the Right Internal Jugular vein 14, which is readily accessible via percutaneous puncture, and within a few centimeters becomes the SVC 18. Techniques for positioning a catheter within these structures have been a standard part of post medical school training, and do not need review herein. Relevant to this discussion, the Left Internal Jugular Vein 15 also leads to the SVC 18 but does not follow a straight line but rather requires negotiating the brachiocephalic vein 17 which may present technical challenges owing to the sinuous route of this vessel.

In a similar fashion, the IVC 21 can be equally effective in terms of a locus for placement of the EXPO catheter. This would be positioned by entry through either of the femoral veins 19 R, L.

It is important to recognize that combinations of the embodiments presented above can be envisioned and anticipated, and that any combination of the embodiments presented would be considered within the spirit and scope of this application.

The iterations and embodiments are presented in their general format, but it is recognized that others who read and become knowledgeable regarding the inventions presented herein, and in particular those generally skilled in the art may evolve and demonstrate other embodiments which are obvious when viewed in the face of these disclosures; clearly, all similar embodiments and iterations are within the spirit and scope of the invention.

The foregoing is considered as illustrative only of the principle of the invention. Furthermore, since numerous modifications and changes will readily occur to those skilled in the art, it is not considered desirable to limit the invention to the exact construction and operation shown and described.

### ROSTER

a. Ascending Aorta
b. Arch of the Aorta
c. Brachiocephalic artery
d. Left common carotid
e. Left subclavian artery
f. Right common carotid
g. Right subclavian artery
h. Descending thoracic aorta
i. Abdominal aorta
j. Right and left iliac arteries
k. Right subclavian vein
l. Left subclavian vein
m. Right femoral artery, R, and L - left femoral artery
n. Right Internal Jugular vein
o. Left Internal Jugular vein
p. Right Brachiocephalic vein
q. Left Brachiocephalic vein
r. Superior Vena Cava
s. R & L / Femoral Veins
t. R & L / Iliac Veins
u. Inferior Vena Cava
v. Pulmonary artery
w. EXPO reoxygenation system
x. Catheter
y. Outer sheath
z. exchange chamber
aa. Connection tubing
bb. Regulator
cc. Leading end of catheter
dd. Trailing end of catheter
ee. Leading end of connection tubing
ff. Central portion of connection tubing
gg. Trailing end of connection portion
hh. Oxygen input tubing
ii. Nitrogen input tubing
jj. Discharge port
kk. Exchange valve of regulator
ll. Leading end of outer sheath
mm. Shaft of the outer sheath
nn. Trailing end of the outer sheath
oo. Apertures in shaft of outer sheath
pp. Alternative embodiment of outer sheathLeading end of alternative embodiment
qq. Shaft of alternative embodiment
rr. Trailing end of alternative embodiment
ss. Leading end of the exchange chamber
tt. Shaft of the exchange chamber
uu. Corrugations of 47
vv. Trailing end
ww. Slidable panel at trailing end
xx. Control for 50
yy. Cap at leading end
zz. Central gas delivery channel
aaa. Apertures in 53
bbb. Lumen with Blood flowing within the outer sheath
ccc. Alternative embodiment of the inner channel
ddd. Central gas delivery channel of 56
eee. baffle for air exchange
fff. Isthmus from channel 56 to baffles
ggg. Lumen between 56 and 25
hhh. Interior of baffles
iii. Superior row of baffles
jjj. Inferior row of baffles
kkk. Orthogonal row of baffles
lll. Alternative embodiment of exchange chamber with larger spheres
mmm. Multiple spheres centered around the central delivery channel
nnn. Central delivery channel
ooo. Apertures connecting 66 and 67
ppp. Alternative embodiment with longitudinal circumferential chambers
qqq. Longitudinal circumferential chambers
rrr. Central gas delivery channel
sss. Trailing end of 71
ttt. Trailing end of 67 in FIG. 10
uuu. Manifold at the trailing end of 71
vvv. Lumen of the catheter in FIGS. 11 & 12
www. Alternative embodiment of catheter in FIG. 13
xxx. Outer sheath of alternative embodiment in 76
yyy. exchange chamber of 76
zzz. Balloon at the leading end of 77.
aaaa. Balloon at the trailing end of 77
bbbb. Lumen in 76
cccc. Tube to inflate 79
dddd. Tube to inflate 80
eeee. Trailing end of 78
ffff. Junction of 27 and 84
gggg. Oxygen intake valve on Regulator
hhhh. Nitrogen intake valve on Regulator
iiii. Oxygen transfer tubing
jjjj. Nitrogen transfer tubing
kkkk. Composition chamber
llll. Oxygen transfer tubing valve
mmmm. Nitrogen transfer tubing valve
nnnn. Oxygen regulation control
oooo. Nitrogen regulation control
pppp. Exiting gas conduit
qqqq. Input reservoir
rrrr. Input transfer tubing
ssss. reservoir output tubing
tttt. Right arm
uuuu. Right a /b tributaries to the right antecubital vein
vvvv. Right Antecubital vein
wwww. Introducing needle
xxxx. Syringe
yyyy. Trailing end of 102
zzzz. Flashback of blood into 103
aaaaa. Guide wire
bbbbb. Trailing end of guide wire
ccccc. Introducer
ddddd. Leading end of 108
eeeee. Trailing end of 108

What is claimed is:

1. A complex intravascular catheter for oxygenating blood comprising
an outer sheath intravascular catheter with a leading end, a central shaft, and a trailing end.
and an exchange chamber configured to be positioned within the outer sheath / catheter.
an exchange chamber substantially fitted within the said catheter, and which is semipermeable, and highly permeable to small molecular compounds including N₂, O₂ and O₂. This establishes the exo-pulmonary oxygenation forming a gas exchange chamber.

2. The complex intravascular catheter of claim 1 wherein said gas exchange chamber has a leading end, a central shaft, and a trailing end.

3. The complex intravascular catheter of claim 2 wherein said gas exchange chamber the central shaft is provided with at least one set of multiple corrugations which substantially increase the surface area.

4. The complex intravascular catheter of claim 1 further comprising a first and a second gas supply passage and connecting a gas supply to said gas exchange chamber.

5. The complex intravascular catheter of claim 4 further comprising a first gas pressure valve connected to said first gas supply line and a second gas pressure valve connected to said second gas supply line.

6. The complex intravascular catheter of claim 5 further comprising an oxygen supply to said first gas supply line.

7. The complex intravascular catheter of claim 6 further comprising a gas supply of nitrogen to said second gas supply line.

8. The complex intravascular catheter of claim 4 further comprising a supply of air to said first gas supply line.

9. The complex intravascular catheter of claim 7 wherein said gas exchange chamber is divided into a first gas exchange portion and a second gas exchange portion that are separated from one another by a passage that allows for flow of a liquid.

10. The complex intravascular catheter of claim 3 further comprises a gas supply line to said multiple corrugations.

11. The complex intravascular catheter of claim 10 further comprising a gas pressure valve connected to said gas supply line.

12. The complex intravascular catheter of claim 11 further has a gas pressure measurement sensor.

13. The complex intravascular catheter of claim 12 further provides a gas flow mechanism for supplying gas and exhausting gas from said gas exchange chamber.

14. The complex intravascular catheter of claim 13 wherein the intake valves for each of the gases (O₂, N₂) is actuated by a pressure sensor within the respective chambers within which of these gases are housed.
